(19) United States
(12) Patent Application Publication
(10) Pub. No.: US 2007/0282421 A1
(43) Pub. Date: Dec. 6, 2007

(54) Stent Assembly for Protecting the Interior Surface of a Vessel

(76) Inventors: Fred T. Parker, Unionville, IN (US); Mark A. Magnuson, Bloomington, IN (US)

Correspondence Address: BRINKS HOFER GILSON & LIONE/CHICAGO/COOK, PO BOX 10395, CHICAGO, IL, 60610, US

(21) Appl. No.: 11/756,265

(22) Filed: May 31, 2007

Related U.S. Application Data
(60) Provisional application No. 60/809,597, filed on May 31, 2006.

(51) Int. Cl. A61F 2/84; A61F 2/94

(52) U.S. Cl. 623/1.12; 623/1.13

(57) ABSTRACT

A method and device includes advancing an anchor stent assembly and a second stent into a stenosed region of a blood vessel to protect or shield the vessel from possible blockage. The anchor stent assembly may include an anchor stent and a cover made of submucosal tissue (SIS). The cover is delivered in a dried, compressed, condition and is later softened and expanded by exposure to the flow of blood. The second stent may then be advanced within the cover and dilated to press the cover against the stenosed region of the vessel, thus protecting the vessel from emboli formed of stenosis breakage.

The present application claims priority to U.S. Provisional Application No. 60/809,597, filed on May 31, 2006.

### BACKGROUND

The present invention relates generally to devices, methods and systems for vascular treatment. One embodiment of the device includes an anchor stent assembly using submucosal tissue to shield at least a portion of the interior surface of a vessel wall from the flow of blood. The device may ideally prevent emboli from a stenosed region of a vessel from flowing down stream and causing clots or other vascular complications.

Vascular diseases and disorders are widespread health problems affecting many people. There are many chronic and acute diseases and disorders relating to the vascular system including, for example, thrombosis, embolism, aneurysm, atherosclerosis, arterioscholerosis, infarction and still others.

Heart attacks and strokes are leading health concerns. Obstruction of blood flow and/or vessel rupture may cause inadequate blood supply to the heart, brain, and other parts or all of the body. Occlusive diseases involving constriction, narrowing or obstruction of a blood vessel often present serious, possibly life-threatening risks. Additionally, complications in vascular treatment(s) may themselves necessitate further treatment. Some such risks include formation of emboli, vessel damage, thrombogenesis, blood loss, hemorrhage, and others. Furthermore, trauma and other injuries may damage the vascular system and often require repair or replacement.

At present, treatment of vascular disease, damage and disorders suffers from limitations, drawbacks and risks. The invention provides unique treatments and solutions to treatment of the foregoing and other problems.

### BRIEF SUMMARY

The endovascular device described below may overcome the aforementioned problems and relates to a medical device, and more particularly, to an endovascular device assembly and method of making the same that shields at least a portion of the interior surface of an organ from emboli and interior breakage.

One embodiment includes a method for shielding at least a portion of the interior surface of an organ, comprising: introducing a device into an organ, the device including an anchor stent assembly and a second stent; wherein the anchor stent assembly has an anchor stent having a proximal end, a distal end, an exterior surface, and an interior lumen, the exterior surface being at least partially covered with a cover comprised of bioremodelable or bioabsorbable material; the cover being in the form of a rod extending beyond the distal end and having an initial diameter; and wherein the second stent has a proximal end, a distal end, an exterior surface, and an interior lumen. The method further includes positioning the device within a specified region of the organ, the distal end of said second stent being positioned approximately adjacent the proximal end of the anchor stent and exposing the cover to the flow of blood; expanding the anchor stent and the cover within the organ; advancing the second stent through the interior lumen of the anchor stent and an interior surface of the cover; and expanding the second stent.

The method as described above, wherein the cover is an extracellular matrix.

The method as described above, wherein the cover is comprised of submucosa dried and crimped into a stiffened rod.

The method as described above, wherein the specified region of the organ is a blood vessel afflicted with a stenosis.

The method as described above, wherein the introducing step includes loading the device into a delivery system comprised of an outer sheath and an inner catheter.

The method as described above, wherein the cover is exposed to the flow of blood when an outer sheath is withdrawn toward the proximal end of the blood vessel.

The method as described above, wherein the anchor stent is a self expanding stent, and wherein the anchor stent expands when an outer sheath is withdrawn toward said proximal end of said blood vessel to expose said anchor stent.

The method as described above, wherein the second stent is a self expanding stent, and wherein the second stent is at least partially expanded upon withdraw of an outer sheath.

The method as described above, wherein the second stent is a self expanding stent, the second stent being at least partially expanded upon withdraw of said outer sheath, and wherein said method further includes post-dilating said second stent with an inflation device.

The method as described above, wherein the inflation device is a balloon catheter.

Another embodiment includes a device for shielding at least a portion of the interior surface of an organ, comprising: an anchor stent assembly and a second stent; the anchor stent assembly having an anchor stent with a proximal end, a distal end, an exterior surface, and an interior lumen, the exterior surface being at least partially covered with a cover comprised of bioremodelable or bioabsorbable material; the cover being in the form of a rod extending beyond said distal end and having an initial diameter; and the second stent having a proximal end, a distal end, an exterior surface, and an interior lumen. The distal end of the second stent is positioned approximately adjacent the proximal end of the anchor stent.

The method as described above, wherein the stiff cover may be softened by exposure to a liquid.

The method as described above, wherein the liquid is blood.

The method as described above, wherein the device further includes a delivery system, the delivery system having an outer sheath and an inner catheter.

The method as described above, wherein the anchor stent assembly and the second stent are disposed between the outer sheath and the inner catheter; the outer sheath being movable relative to the inner catheter.

The method as described above, wherein the cover is an extracellular matrix.

The method as described above, wherein the cover is comprised of submucosa dried and crimped into a stiffened rod.

Yet another embodiment includes a method for shielding at least a portion of the interior surface of an organ, comprising: introducing an anchor stent assembly into said organ, wherein the anchor stent assembly includes an anchor stent having a proximal end, a distal end, an exterior surface, and an interior lumen, said exterior surface being at least partially covered with a cover comprised of bioremodelable or bioabsorbable material, the cover being in the form of a rod extending beyond the distal end and having an initial diameter; positioning the anchor stent assembly within a specified region of said organ; exposing the cover to the flow of blood; expanding the anchor stent within the organ; introducing a second stent into said organ, the second stent having a proximal end, a distal end, an exterior surface, and an interior lumen; advancing the second stent through the interior lumen of the anchor stent and an interior surface of the cover; and expanding the second stent.

The method as described above, wherein the anchor stent assembly and said second stent are disposed within a single delivery device and said distal end of the second stent is positioned approximately adjacent to the proximal end of the anchor stent.

The method as described above, wherein the anchor stent assembly and the second stent are disposed within separate delivery devices.

Another embodiment includes an anchor stent assembly, comprising: an anchor stent having a proximal end, a distal end, an exterior surface, and an interior lumen, the exterior surface being at least partially covered with a cover comprised of bioremodelable or bioabsorbable material, the cover being in the form of a rod extending beyond the distal end and having a first initial diameter less than an expanded diameter of the proximal end, the cover constraining the distal end to a second initial diameter less than the expanded diameter of the proximal end.

The method as described above, wherein the first initial diameter and the second initial diameter are equal.

The method as described above, wherein the first initial diameter is less than the second initial diameter.

The method as described above, wherein the cover is an extracellular matrix.

The method as described above, wherein the cover is comprised of submucosa dried and crimped into a stiffened rod.

### BRIEF DESCRIPTION OF THE DRAWINGS

FIG. 1 is a side view of a stenosed region of a blood vessel;

FIG. 2 is a side view of one example of an anchor stent and rod-like covering material

FIG. 3 is a side view of one embodiment of a pre-deployment stent assembly;

FIG. 4 is a side view of a stenosed region of a blood vessel;

FIG. 5 is a side view of a stent assembly advanced through a stenosed region of a blood vessel;

FIG. 6 is a side view of a stent assembly;

FIG. 7 is a side view of a stent assembly advanced through a stenosed region of a blood vessel;

FIGS. 8A and 8B are a series of side views showing a method for using the delivery system;

FIG. 9 is a side view of a stent assembly advanced through a stenosed region of a blood vessel; and

FIG. 10 is a side view of a stent assembly advanced through a stenosed region of a blood vessel.

### DETAILED DESCRIPTION

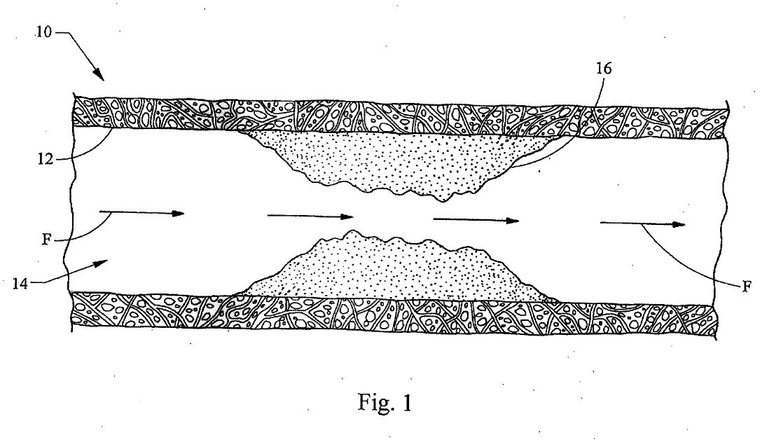

A device and method for protecting and reinforcing blood vessels afflicted with stenosis is shown in FIGS. 1-10. The device and method include the use of small intestine submucosa (SIS) or other suitable material. The device and method further include the use of at least two structural stents, supporting the SIS material against a stenosed region.

With reference to FIG. 1, there is shown an illustrative view of a blood vessel 10 which includes an interior surface 12 and a lumen 14. Blood flow through the vessel 10 is generally in the direction indicated by arrow F and may vary according to physiological conditions.

Stenosis is a narrowing or constriction of the lumen of the vessel 10 in the region generally indicated at 16. The narrowing or constriction of the lumen may result in reduced blood flow through the vessel 10 and may increase the risk of thrombosis, embolism, and other complications. While stenosis 16 is illustrated, other diseases, damage, or disorders could be present in region 16, or in other regions. For example, thrombosis, aneurysm, lodged embolism, necrotic tissue, cut or damaged vessel 10 tissue, perforation, and other lesions, disease, disorders or damage may all be treated by the present invention. For the sake of brevity, treatment of stenosis 16 is illustrated and described with the understanding that treatment of the aforementioned diseases and others is also contemplated and protected.

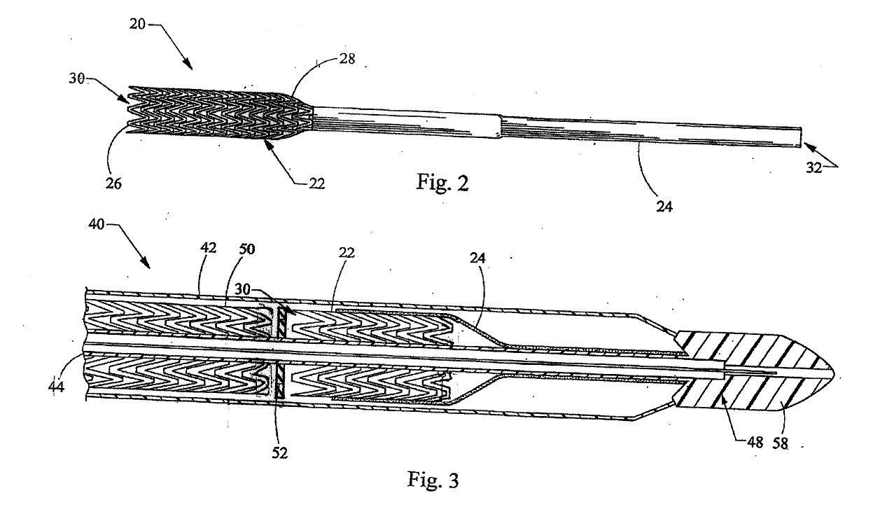

With reference to FIG. 2, there is shown an anchor stent assembly 20, including anchor stent 22 having an exterior surface 28 partially covered by a rod-like cover 24. Reconstituted or naturally-derived collagenous materials can be used for the covering material in the present invention. Such materials that are at least bioresorbable will provide advantage in the present invention, with materials that are bioremodelable and promote cellular invasion and ingrowth providing particular advantage.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

By way of example, this application will refer to SIS as the cover 24, but other ECM materials are contemplated. SIS may generally be obtained in tubular form and may come in at least one of two forms, vacuum pressed or liotholized. The vacuum pressed SIS is almost translucent in appearance and very rigid. The liotholized SIS is generally freeze dried and much more flexible and pliable than the vacuum pressed variation.

The SIS cover 24 may be wetted in order to make it flexible and malleable. Once the cover 24 is wetted, it is mounted on an expanded anchor stent 22. The anchor stent 22 may be any suitable stent, but desirably will be a self expandable stent. Examples of appropriate stents are disclosed in U.S. Pat. No. 4,580,568 to Gianturco, U.S. Pat. No. 5,928,280 to Hansen et al., and U.S. Pat. No. 5,968,088 to Hansen et al., and are incorporated herein by reference. The anchor stent 22 may include an interior surface 26, an exterior surface 28, and an inner lumen 30. As shown in FIG. 2, approximately half of the anchor stent 22 may be covered with the SIS cover 24. For example, a 2 cm stent may have approximately 1 cm of its exterior surface covered with the SIS cover. The length of the anchor stent 22 and of the SIS cover 24 may vary based upon the affliction being treated and the patient's own body size.

The expanded anchor stent 22 and cover 24 may then be crimped over a mandrel which can be inserted through the central lumen 30, 32 of both the stent 22 and cover 24. The mandrel around which the stent 22 and the cover are crimped may be as small as 0.014 inches in diameter, but may be larger depending upon the desired size of the resulting partially covered compressed anchor stent 22. The mandrel may generally be used to provide a center lumen 30, 32 within the resulting anchor stent 22 and cover 24.

Once the mandrel is inserted into the anchor stent assembly 20, a crimping machine, or other suitable method, may be used to compress the anchor stent 22 and cover 24, decreasing the outer diameter of the stent assembly 20. By crimping the cover 24 around the anchor stent 22, it is contemplated that the stent 22 and the cover 24 will have a decreased diameter that may be later expanded to their initial size. The crimping process may be carried out in a variety of ways, including manually and mechanically.

The inner lumen 30, 32 of the anchor stent 22 and the cover 24 allow the practitioner to position the assembly 20 on an inner catheter and guidewire of a delivery system (discussed below).

The SIS cover 24 is dried while in the crimping machine. The resulting material may be stiff and rod-like. This may enable the practitioner to more easily manipulate the cover 24 within the delivery system and the patient's body. One method of drying the cover 24 is to use a hot air gun; however other methods such as air drying have also been contemplated. After the cover 24 is dried in a compressed state, it will form a resilient rod-like tube, holding the self-expanding anchor stent 22 at a decreased diameter.

Once assembled, the anchor stent 22 and rod-like cover 24 may be transferred to a delivery device 40. The transfer may be effectuated by placing the anchor stent assembly 20 in a transfer tube and pushing the assembly 20 into the device 40. Additionally, it may be possible to take the cover 24 and anchor stent 22 directly from the crimping machine to the delivery device 40. One advantage of using a transfer tube is that it holds the delivery device 40 and anchor stent assembly 20 in the correct position and helps to position the anchor stent assembly 20 within the delivery device 40.

Any suitable delivery device may be used; however, it is desirable that the device include an outer sheath 42 and an inner catheter 44. One such delivery device is disclosed in U.S. Pat. No. 5,700,253 to Parker, which is incorporated herein by reference.

The anchor stent assembly 20 is loaded into the delivery device 40 over the inner catheter 44 in a compressed condition. Generally, the assembly 20 may be loaded into the delivery device 40 from the device's proximal end 46. This may prevent or lessen the chance that the rod-like SIS cover 24 will be broken during the loading process. However, the assembly 20 could be loaded into the delivery device 40 from either end using known techniques.

As shown in FIG. 3, after the anchor stent assembly 20 is loaded into the delivery device 40 and positioned toward the distal end 48 of the device 40, a second stent 50 may also loaded into the device 40, behind the anchor stent assembly 20. The second stent 50, like the anchor stent 22, may generally be a self expandable vascular stent. However, the second stent 50 could also be a balloon expandable vascular stent, or any other structure that is not classified as a stent but capable of being introduced into a blood vessel and which can maintain at least a portion of the SIS cover 24 in a desired position or location.

The second stent 50 may be loaded into the delivery device 40 similarly to the anchor stent 22, compressed over the inner catheter 44. Generally, the outer sheath 42 of the delivery device 40 covers the second stent 50, the anchor stent assembly 22, and the cover 24, preventing premature expansion of the stents.

The inner catheter 44 may include markers or pushers 52. The pushers 52 have generally the same outer diameter as the compressed stents 22, 50. The pushers, however, may have an inner diameter that is less than the outer diameter of the tip 58, preventing the pusher 52 from separating from the inner catheter 44. In order to effectively launch both stents 22, 50 into the desired vessel 10, the pushers 52 may be located on or around the inner catheter 44, between the anchor stent 22 and the second stent 50, as shown in FIG. 3. A second set of markers and/or pushers 54 may also be located behind the second stent 50. One suitable marker would be a radiopaque ring surrounding the inner catheter 44 with an outer diameter large enough to impede the anchor stent 22 and the second stent 50 in their compressed condition, but small enough to pass through the central lumen 30, 56 of the stents 22, 50 when they are expanded or deployed within the vessel 10.

The inner catheter 44 may also include a tip 58. The tip 58 could be a flexible tip, a guiding tip, a cannula or another tip or tips of differing size, shape, and structure. The tip 58 may generally be made of a soft material, such as polyurethane, and may be attached only to the inner catheter 44. In another embodiment, the tip 58 may also have part of the SIS cover 24 tucked under, or removably attached to, the tip 58. This embodiment may lessen the concern that the dried rod-like cover 24 will break upon insertion into the delivery device 40.

Referring still to FIG. 3, the outer sheath 42 maintains the anchor stent 22 and the second stent 50 in a compressed state in the case of a self-expanding stent, for example. The inner catheter 44, the anchor stent assembly 22, the cover 24, and the second stent 50 may be enclosed within the outer catheter 42. This allows the practitioner to use both stents 22, 50 without performing multiple procedures. In some embodiments more than one subsequent stent may be enclosed within the outer catheter. This may allow the practitioner to treat multiple stenosed regions or regions that vary in length and severity.

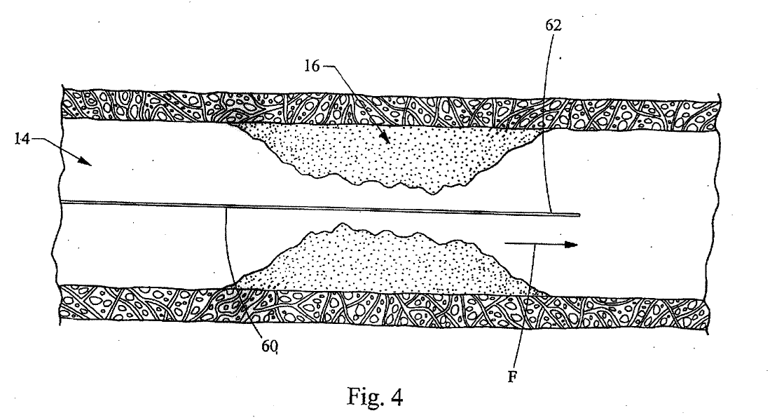

Referring now to FIG. 4, one method of introducing the loaded delivery device 40 into the body is to insert a guidewire 60 into the vessel lumen 14 of the patient. The guidewire 60 is positioned within the organ to be treated with its distal end advanced, in the direction indicated by arrow F, through and past the distal end of the stenosed region 16. Generally, the guidewire 60 may be as small as 0.014 inches in diameter.

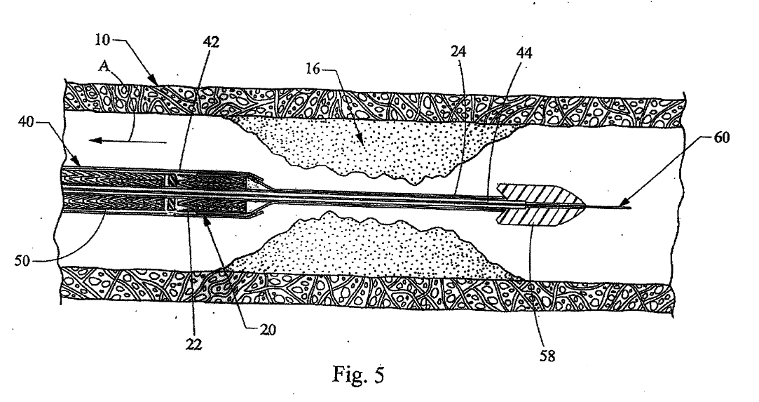

As shown in FIG. 5, once the guidewire 60 is positioned within the vessel 10, the delivery device 40, including the anchor stent assembly, 22, the cover 24, and the second stent 50, is threaded over the guidewire 60. The tip 58 of the inner catheter 44, as well as the distal end of the outer sheath 42, may include a radiopaque marker used for positioning purposes. In this manner, the tip 58 of the inner catheter 44 can be positioned distally of the stenosed region 16 of the vessel 10 to be treated. The anchor stent 22 and the second stent 50 may also include radiopaque markers at either end. The markers may be in the form of gold rivets on the terminating eyelets of the stent bodies.

Referring again to FIG. 5, the cover 24 may be positioned through the stenosed region 16 of the vessel 10. The low profile of the crimped, rod-like material allows the cover 24 to cross legions or stenosis 16, minimizing disturbance to the area. The cover 24 may be long enough to protect the entire length of the stenosis 16 to be treated.

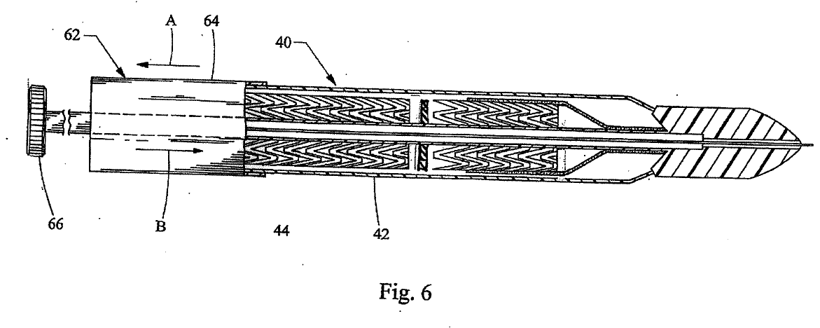

One example of a deployment method is shown in FIGS. 6-10. The proximal end of the delivery device, including the outer sheath 42 and the inner catheter 44, may be attached to a handle 62. The handle 62 is generally located outside of the patient's body and allows the sheath 42 and the inner catheter 44 to be moved independently of, or relative to, one another.

In one embodiment, a tube-like structure 64 may be attached to the proximal end of the sheath 42, allowing the user to withdraw the outer sheath 42 in the direction of arrow A. In addition, the inner catheter 44 may be attached to a back bushing 66 and may be disposed through the tube-like structure 64. The user may be able to hold the inner catheter 44 in place, while the tube-like structure 64 pulls the outer sheath 42 in the direction of arrow A. Alternatively, the inner catheter 44 may be moved in the direction of arrow B by moving the inner catheter 44 in and out of the tube 64, holding the back bushing 66 for support.

In FIG. 5, the loaded delivery device 40 has been moved into the deployment position. Once the loaded delivery device 40 is in place, using the handle 62 (FIG. 6), the user may begin to withdraw the outer sheath 42 relative to the anchor stent assembly 22 and the second stent 50, in the direction of arrow A. Other deployment techniques and devices are also contemplated.

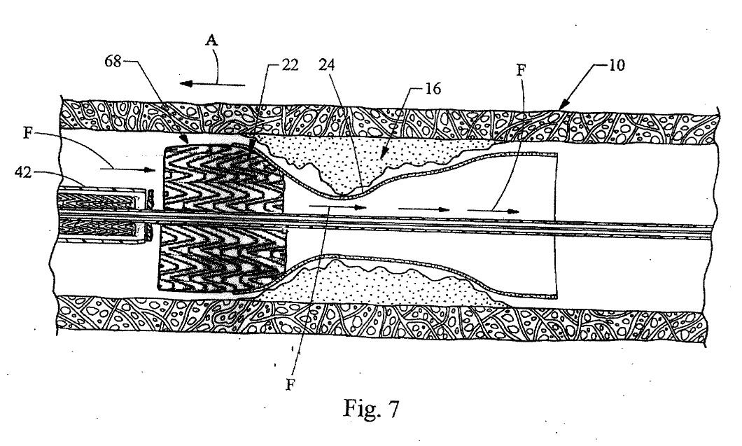

Regardless of which deployment mode is used, as shown in FIG. 7, when the outer sheath 42 is withdrawn in the direction of arrow A, the cover 24 and the anchor stent 22 are exposed to the flow of blood. The proximal end 68 of the anchor stent 22 may expand as it exits the outer sheath 42. As the flow of blood softens the rod-like cover 24, the hemostatic pressure of the blood flow and the expanding anchor stent 22 may cause the cover 24 to expand with the anchor stent 22 and against the stenosed region 16 of the vessel 10. The cover 24 can conform to the shape of the interior of the vessel 10 as well as to the irregularities presented by the stenosis 16. The cover 24 may be temporarily held against the distal end of the stenosed region 16 by the vessel's natural hemostatic pressure. Hemostatic pressure may be present within the cover 24 due to the flow of blood therethrough, but in the case of trauma or patient and treatment conditions, for example, wide variation in pressure may exist. Blood flow generally enters the cover 24 through the proximal end and is routed through the inner lumen of the cover and out of the distal end, in the direction indicated by arrow F. Thus, blood flow is isolated from the stenosis 16.

After the anchor stent 22 has been deployed, the practitioner will ideally wait about one minute when this procedure is performed on a patient with good blood flow through the stenosed region. The time allotted for the cover 24 to soften may vary from about 30 seconds to about 2 minutes, depending on the patient and the blood pressure through the region.

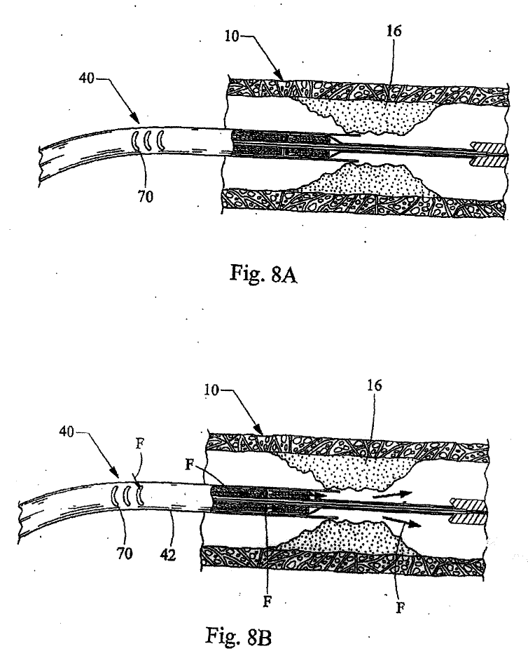

When the loaded delivery device 40 is inserted into the stenosed region 16 of the blood vessel 10, it may temporarily cut off blood flow through the vessel 10. (See FIG. 5). Therefore, holes or slits 70 may be cut or put in the outer sheath 42 so that the blood can flow through the outer sheath 42, as shown in FIGS. 8a and 8b.

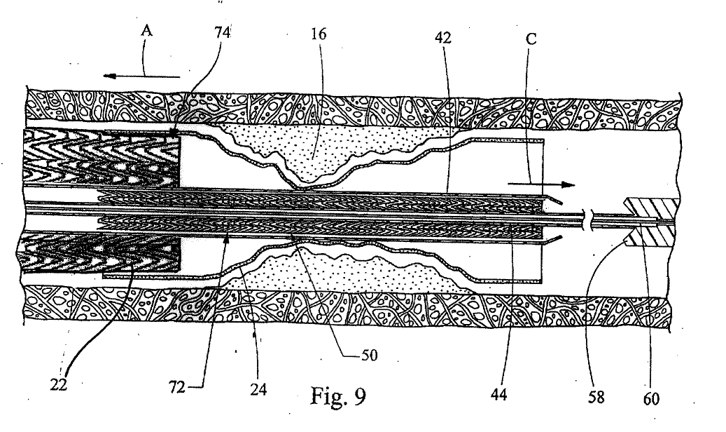

With reference to FIG. 9, there is illustrated deployment of the second stent 50. In FIG. 9, the guidewire 60 has been advanced in the direction indicated by arrow C, which is effective to move the tip 58 of the inner catheter 44 in the same direction. The result of this movement is to position the second stent 50 within the stenosed region 16, with the proximal end 72 of the second stent 50 generally overlapping with the distal end 74 of the anchor stent 22. The positions of the ends of the stents may generally be obtained by using radiopaque markers disposed at either end. The second stent 50 may generally extend the length of the stenosed region 16 and may or may not extend past the distal end of the cover 24.

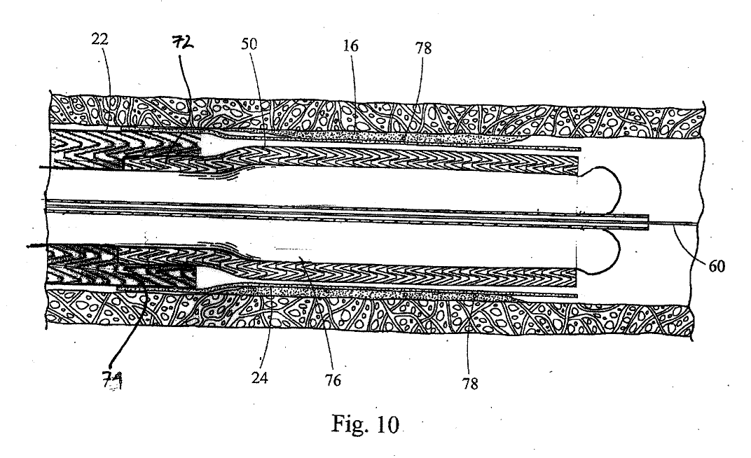

Once the second stent 50 has been advanced through the stenosis 16, the outer sheath 42 may again be withdrawn in the direction of arrow A, thus exposing the second stent 50 to the inner surface of the cover 24. Again, the self expanding second stent 50 expands as it exits the sheath 42. The delivery device, including the outer sheath 42 and inner catheter 44, may then be removed from the patient's body. As shown in FIG. 10, upon removal of the delivery device, a post-dilation balloon catheter 76 may then be inserted into the stenosed region 16 to fully expand the second stent 50, the cover, and the anchor stent against the vessel wall. This expansion exerts force on the stenosis 16 which may cause it to break down. This treatment and others can produce emboli 78 which are fragments of the stenosis 16. The cover 24 protects the emboli 78 from blood flow and may prevent them from entering the blood stream. Generally, the practitioner will sequentially dilate the balloon, expanding the balloon gradually to fully open the stenosed region, beginning at the distal end of the vessel 10.

In another embodiment, the second stent 50 may be introduced separately from the anchor stent assembly 20. In this embodiment, the anchor stent assembly 20 may be loaded into the delivery device 40, as described above. The anchor stent assembly may be inserted in to the vessel 10 and deployed, as described above. The assembly 20 may be deployed within the vessel 10 by positioning the cover 24 through the stenosed region 16 of the vessel 10 and the outer sheath 42 is withdrawn, relative to the anchor stent assembly. The cover 24 and the anchor stent 22 are exposed to the flow of blood and the cover is softened. At this point, the delivery device 40 may be withdrawn from the vessel 10, leaving the anchor stent assembly 20 deployed within the stenosed region. The second stent 50 may then be deployed within the vessel by known techniques.

It is intended that the foregoing detailed description be regarded as illustrative rather than limiting, and that it be understood that it is the following claims, including all equivalents, that are intended to define the spirit and scope of this invention.

1. A method for shielding at least a portion of the interior surface of an organ, comprising:
introducing a device into an organ, the device including an anchor stent assembly and at least a second stent;
wherein said anchor stent assembly has an anchor stent having a proximal end, a distal end, an exterior surface, and an interior lumen, said exterior surface being at least partially covered with a cover comprised of bioremodelable or bioabsorbable material;
said cover being in the form of a rod extending beyond said distal end and having an initial diameter; and
wherein said second stent has a proximal end, a distal end, an exterior surface, and an interior lumen;
positioning said device within a specified region of said organ, said distal end of said second stent being positioned approximately adjacent said proximal end of said anchor stent;
exposing said cover to the flow of blood;
expanding said anchor stent and said cover within said organ;
advancing said second stent through said interior lumen of said anchor stent and an interior surface of said cover; and
expanding said second stent.

2. The method of claim 1, wherein said cover is an extracellular matrix.

3. The method of claim 1, wherein said cover is comprised of submucosa dried and crimped into a stiffened rod.

4. The method of claim 1, wherein said specified region of said organ is a blood vessel afflicted with a stenosis.

5. The method of claim 1, wherein said introducing step includes loading said device into a delivery system comprised of an outer sheath and an inner catheter.

6. The method of claim 1, wherein said cover is exposed to the flow of blood when an outer sheath is withdrawn toward said proximal end of said blood vessel.

7. The method of claim 1, wherein said anchor stent is a self expanding stent, and wherein said anchor stent expands when an outer sheath is withdrawn toward said proximal end of said blood vessel to expose said anchor stent.

8. The method of claim 1, wherein said second stent is a self expanding stent, and wherein said second stent is at least partially expanded upon withdraw of an outer sheath.

9. The method of claim 1, wherein said second stent is a self expanding stent, said second stent being at least partially expanded upon withdraw of said outer sheath, and wherein said method further includes post-dilating said second stent with an inflation device.

10. The method of claim 9, wherein the inflation device is a balloon catheter.

11. A device for shielding at least a portion of the interior surface of an organ, comprising:
an anchor stent assembly and a second stent;
said anchor stent assembly having an anchor stent with a proximal end, a distal end, an exterior surface, and an interior lumen, said exterior surface being at least partially covered with a cover comprised of bioremodelable or bioabsorbable material;
said cover being in the form of a rod extending beyond said distal end and having an initial diameter; and
said second stent having a proximal end, a distal end, an exterior surface, and an interior lumen; and
said distal end of said second stent being positioned approximately adjacent said proximal end of said anchor stent.

12. The device of claim 11, wherein said stiff cover may be softened by exposure to a liquid.

13. The device of claim 12, wherein said liquid is blood.

14. The device of claim 11, wherein said device further includes a delivery system, said delivery system having an outer sheath and an inner catheter.

15. The device of claim 14, wherein said anchor stent assembly and said second stent are disposed between said outer sheath and said inner catheter; said outer sheath being movable relative to said inner catheter.

16. The device of claim 11, wherein said cover is an extracellular matrix.

17. The device of claim 11, wherein said cover is comprised of submucosa dried and crimped into a stiffened rod.

18. A method for shielding at least a portion of the interior surface of an organ, comprising:
introducing an anchor stent assembly into said organ, wherein said anchor stent assembly includes an anchor stent having a proximal end, a distal end, an exterior surface, and an interior lumen, said exterior surface being at least partially covered with a cover comprised of bioremodelable or bioabsorbable material, said cover being in the form of a rod extending beyond said distal end and having an initial diameter;
positioning said anchor stent assembly within a specified region of said organ;
exposing said cover to the flow of blood;
expanding said anchor stent within said organ;
introducing a second stent into said organ, said second stent having a proximal end, a distal end, an exterior surface, and an interior lumen;
advancing said second stent through said interior lumen of said anchor stent and an interior surface of said cover; and
expanding said second stent.

19. The method of claim 16, wherein said anchor stent assembly and said second stent are disposed within a single delivery device and said distal end of said second stent is positioned approximately adjacent to said proximal end of said anchor stent.

20. The method of claim 16, wherein said anchor stent assembly and said second stent are disposed within separate delivery devices.

21. An anchor stent assembly, comprising:
an anchor stent having a proximal end, a distal end, an exterior surface, and an interior lumen, said exterior surface being at least partially covered with a cover comprised of bioremodelable or bioabsorbable material, said cover being in the form of a rod extending beyond said distal end and having a first initial diameter less than an expanded diameter of said proximal end, said cover constraining said distal end to a second initial diameter less than said expanded diameter of said proximal end.

22. The device claim of 21, wherein said first initial diameter and said second initial diameter are equal.

23. The device of claim 21, wherein said first initial diameter is less than said second initial diameter.

24. The device of claim 21, wherein said cover is an extracellular matrix.

25. The device of claim 21, wherein said cover is comprised of submucosa dried and crimped into a stiffened rod.
